(19) United States
(12) Patent Application Publication
(10) Pub. No.: US 2008/0099017 A1
(43) Pub. Date: May 1, 2008

(54) Modular positive airway pressure therapy apparatus and methods

(76) Inventors: Steven S. Bordewick, Minneapolis, MN (US); Bruce Bowman, Eden Prairie, MN (US); Joseph A. Baser, Lino Lakes, MN (US)

Correspondence Address: CYR & ASSOCIATES, P.A., 605 U.S. Highway 169, Suite 300, Plymouth, MN, 55441, US

(21) Appl. No.: 11/906,389

(22) Filed: Oct. 1, 2007

Related U.S. Application Data
(63) Continuation of application No. PCT/US07/14295, filed on Jun. 18, 2007.
(60) Provisional application No. 60/814,636, filed on Jun. 16, 2006.

(51) Int. Cl. A61M 16/00

(52) U.S. Cl. 128/204.21

(57) ABSTRACT

Respiratory therapy apparatus and methods to deliver one or more positive airway pressure therapies are disclosed. The respiratory apparatus includes a flow generator having a first interlock and at least one module that includes a second interlock. The first interlock and the second interlock releasably engagable with one another. The flow generator and the module may be secured in a stacked vertical configuration.

### CROSS REFERENCE TO RELATED APPLICATIONS

This application is a continuation of international application number PCT/US07/14295 filed 18 Jun. 2007 which designates the U.S., and, in turn, claims priority and benefits of U.S. Provisional Application Ser. No. 60/814,636 filed on Jun. 16, 2006. The disclosure of U.S. Provisional Application Ser. No. 60/814,636 is hereby incorporated herein by reference in its entirety.

### BACKGROUND OF THE INVENTION

### Summary of the Invention

The present inventions relate to respiratory therapy and, more particularly, to modular apparatus for providing positive airway pressure therapy and methods for interconnecting modules of modular positive airway pressure apparatus.

### DESCRIPTION OF THE RELATED ART

Respiratory therapy devices typically deliver pressurized air including air and other breathable gasses to a user by way of the nose and/or mouth in order to prevent upper airway occlusion during sleep. The pressurized air is typically administered to a user through a user interface. The user interface is typically secured about the user's head and/or face and configured to deliver pressurized air for inhalation by the user. The pressurized air is typically delivered to the user at pressures ranging between about 4 cm to 20 cm of water. Respiratory therapy devices have become the devices of choice for the treatment of chronic sleep apnea, chronic pulmonary obstruction, and snoring. Many variations of respiratory therapy devices are now commercially available.

A typical respiratory therapy device includes a flow generator, a delivery tube and a user interface. The flow generator often includes a fan unit powered by an electric motor to generate pressurized air. The flow generator is typically configured to deliver pressurized air to the user interface via the delivery tube. The user interface can be secured about the head and face of the user so that the user may breathe the pressurized air.

The respiratory therapy device is often used in a bedroom, so that minimizing the size of the respiratory therapy device may be important. In particular, it may be important to minimize the footprint of the respiratory therapy device so that the respiratory therapy device may fit on a nightstand or small table adjacent to the user's bed. Minimizing size and footprint also increases portability and the ease of use of the respiratory therapy in other environments such as campers, motel rooms, and other spatially restricted locations.

Some source of electrical power must be provided to operate the respiratory therapy device. Because users may wish to utilize the respiratory therapy device in locations lacking electrical power, the ability to operate a respiratory therapy device using battery power may also be important. Also, the ability to humidify the pressurized air delivered by the respiratory therapy device may be important for some users, while other users may not require humidity. In certain applications, the ability to download digital data from the respiratory therapy device and the ability to upload digital data to the respiratory therapy device may also be important. For example, memory within the respiratory therapy device may accumulate data related to use of the respiratory therapy device. This data may be downloaded from the respiratory therapy device and sent for review. Following review of the information, it may be determined that the operation of the respiratory therapy device may require adjustment. Therefore, the ability to upload data to the respiratory therapy device may be important.

While a respiratory therapy device may include a variety of attributes such as, for example, those cited above, not all attributes are necessarily required under all operational conditions. Thus, it may be desirable to be able to configure the respiratory therapy device with the needed attributes while eliminating unneeded attributes, and to be able to change the configuration of the respiratory therapy as the needed attributes change.

### SUMMARY OF THE INVENTION

Apparatus and methods in accordance with the present invention may resolve many of the needs and shortcomings discussed above and provide additional improvements and advantages as will be recognized by those skilled in the art upon review of the present disclosure.

The present invention provides a respiratory therapy apparatus to deliver one or more positive pressure therapies to the user. The respiratory therapy apparatus may include a flow generator. The flow generator may include a microcontroller, a blower, a blower motor, and a first interlock. The microcontroller regulates the operation of at least the blower motor. The respiratory therapy apparatus may further include at least one module having a second interlock secured to the flow generator in a stacked vertical configuration. The first interlock and the second interlock may be configured as male and female so that the male interlock may be slidably or insertably received by the female interlock. The interlocks may also be a combination of male and female components.

When the flow generator is secured to at least one module in a stacked vertical configuration, the flow generator may be in communication with at least one module by a communication interface.

Methods according to the present inventions may include providing a flow generator and a module, and securing the module to the flow generator in a vertical stacked orientation.

Other features and advantages of the invention will become apparent from the following detailed description, and from the claims.

### BRIEF DESCRIPTION OF THE DRAWINGS

FIG. 1A illustrates a perspective view of an exemplary embodiment of a respiratory therapy apparatus in accordance with aspects of the present inventions;

FIG. 1B illustrates another perspective view of an exemplary embodiment of a respiratory therapy apparatus in accordance with aspects of the present inventions;

FIG. 2 illustrates an exploded perspective view of an exemplary embodiment of a respiratory therapy apparatus in accordance with aspects of the present inventions;

FIG. 3 illustrates a perspective view of an exemplary embodiment of a respiratory therapy apparatus in accordance with aspects of the present inventions;

FIG. 4 illustrates an exploded perspective view of an exemplary embodiment of a respiratory therapy apparatus in accordance with aspects of the present inventions;

FIG. 5 illustrates a cross-sectional side view of an exemplary embodiment of a respiratory therapy apparatus of a respiratory therapy apparatus in accordance with aspects of the present inventions;

FIG. 6 illustrates a perspective view of an exemplary embodiment of a respiratory therapy apparatus in accordance with aspects of the present inventions;

FIG. 7 illustrates an exploded perspective view of an embodiment of a respiratory therapy apparatus in accordance with aspects of the present inventions;

FIG. 8A illustrates a rear view of an exemplary embodiment of portions of a respiratory therapy apparatus in accordance with aspects of the present inventions;

FIG. 8B illustrates a bottom view of an exemplary embodiment of portions of a respiratory therapy apparatus in accordance with aspects of the present inventions substantially corresponding to the embodiment illustrated in FIG. 8A;

FIG. 9A illustrates a rear view of an exemplary embodiment of portions of a respiratory therapy apparatus in accordance with aspects of the present inventions;

FIG. 9B illustrates a bottom view of an exemplary embodiment of portions of a respiratory therapy apparatus in accordance with aspects of the present inventions substantially corresponding to the embodiment illustrated in FIG. 9A;

FIG. 10A illustrates an exploded perspective view of an exemplary embodiment of a respiratory therapy apparatus in accordance with aspects of the present inventions;

FIG. 10B illustrates a cross-sectional view of portions of the exemplary embodiment of the respiratory therapy apparatus illustrated in FIG. 10A in accordance with aspects of the present inventions;

FIG. 11 illustrates an exploded perspective view of an exemplary embodiment of a respiratory therapy apparatus in accordance with aspects of the present inventions; and,

FIG. 12 illustrates by block diagram an exemplary embodiment of portions of a respiratory therapy apparatus in accordance with aspects of the present inventions.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “above,” “below,” “superior,” “inferior,” “left,” “right,” “proximal,” “distal,” and similar positional terms are used, the terms should be understood to reference the structures shown in the drawing(s) being described or as they will typically be utilized by a physician administering treatment or user being treated with a respiratory therapy apparatus in accordance with the present inventions.

### DETAILED DESCRIPTION OF THE INVENTION

The present inventions provide respiratory therapy apparatus 10 and associated methods for treatment of sleep apnea and other respiratory and sleeping disorders. The respiratory therapy apparatus 10 are typically configured to communicate pressurized air to a user lying in bed from a remotely positioned flow generator 12. Frequently, the flow generator 12 is remotely positioned on a bedside table, floor or otherwise as will be recognized by those skilled in the art upon review of the present disclosure. The respiratory therapy apparatus 10 may include a flow generator 12, a delivery tube 80, and a user interface 90. The flow generator 12 is typically provided as a source of pressurized air. The delivery tube 80 is generally configured to communicate pressurized air from the flow generator 12 to the user interface 90. The user interface 90 is configured to communicate the pressurized air into the airways of a user. Typically, the user interface 90 is configured to be secured relative to the user's head in communication with the airways of a user to provide a positive pressure therapy as the user sleeps.

The figures generally illustrate exemplary embodiments of respiratory therapy apparatus 10 or components thereof which include aspects of the present inventions. The particular embodiments of the respiratory therapy apparatus 10 illustrated in the Figures have been chosen for ease of explanation and understanding of various aspects of the present inventions. These illustrated embodiments are not meant to limit the scope of coverage but instead to assist in understanding the context of the language used in this specification and the appended claims. Accordingly, many variations from the illustrated embodiments may be encompassed by the appended claims.

Respiratory therapy apparatus 10 in accordance with aspects of the present invention includes a flow generator 12 configured to provide one or more positive airway pressure therapies to the user. The one or more positive airway pressure therapies may include continuous positive airway pressure therapy (CPAP), bi-level positive airway pressure therapy (BPAP), auto positive airway pressure therapy (auto-PAP), proportional positive airway pressure therapy (PPAP), and/or other positive airway pressure therapies as will be recognized by those skilled in the art upon review of this disclosure.

Respiratory therapy apparatus 10 in accordance with the present inventions include at least a flow generator 12 and at least one module 14. One or more modules 14 are typically secured to the flow generator 12. Typically, the modules 14 are releasably secured to the flow generator 12 and/or one another such that the modules 14 and flow generator may be secured and separated by a user without the use of tools. The modules 14 may further be in electrical communication and/or data communication with the flow generator 12. In one aspect, at least one of the modules 14 and the flow generator are stacked in a vertical configuration. When stacked, the adjacent modules 14 may be positioned such that the bottom surface 414 of the module 14 in a superior position is positioned adjacent to or on an upper surface 614 of the module 14 in an inferior position. The upper surface 614 is not necessarily the upper most surface of the module 14. Similarly, a module 14 may be positioned superior to the flow generator 12 such that a bottom surface 414 of the module 14 is positioned adjacent to or on an upper surface 612 of the flow generator 12. Again, the upper surface 614 is not necessarily the upper most surface of the module 14. As illustrated for exemplary purposes, the flow generator 12 may be positioned superior to a module 14 such that the bottom surface 412 of the flow generator 12 is positioned adjacent to or on an upper surface 614 of the module 14.

In certain aspects, respiratory therapy apparatus 10 in accordance with the present inventions may permit the addition of various modules 14 without altering the footprint of the respiratory therapy apparatus 10. The footprint is typically defined as the surface area covered by the flow generator 12 or lowermost module 14 which rests on the supporting surface (i.e. bedside table). In certain configurations, the respiratory therapy apparatus 10 may provide one or more positive airway pressure therapies to the user. The one or more positive airway pressure therapies may include humidification and/or the introduction of medicinal compounds into the flow of pressurized air to the user. In certain configurations, the respiratory therapy apparatus 10 may provide modules 14 for the transfer of data indicative of various aspects of the user's respiratory therapy and may permit the communication of data between two or more modules. The modules may be releasably secured to one another to form a unitary device or may be freestanding components communicating with one another through one or more wires and/or using one or more wireless systems.

Modules 14 may include a humidifier module 16, a data transfer module 18, and a battery module 20, among other modules 14 as will be recognized by those skilled in the art upon review of the present disclosure. While, in certain aspects, at least one module 14 is releasably secured to the flow generator 12, one or more of the functions of various modules may be integrated into the flow generator 12. As such, components from certain modules may be integrated into the housing 40 of the flow generator 12 and operably connected to the components of the flow generator 12.

The humidifier module 16 includes components generally configured to add humidity and/or therapeutic compounds to the pressurized air generated by the flow generator 12. The data transfer module 18 typically includes components generally configured to transfer data indicative of therapeutic data or other data from the respiratory therapy apparatus 10 to a data storage medium or onto computer or a network of computers. The battery module 20 includes components generally configured to provide electrical current to the flow generator 12 and, when present, one or more of modules 14. The battery module 20 may be configured as a primary power source or may be configured as a back-up power supply. When configured as a back-up power supply, the battery module 20 may be configured to maintain a full charge until power is lost from an external power supply. The battery module 20 may then be configured to power the flow generator 12 and any associated modules 14 until power from the external power supply is regained or until the battery of the battery module 20 is discharged.

The flow generator 12 and at least one of the associated modules 14 may each include at least one interlock 30. The interlocks 30 are generally configured to releasably secure adjacent modules 14 to one another or to releasably secure the flow generator 12 to an adjacent module 14. Typically, interlocks 30 on adjacent modules 14 or interlocks 30 on the flow generator 12 and the adjacent module 14 cooperate and/or engage one another to releasably secure the adjacent modules 14 and the module 14 and the flow generator 12, respectively. The interlocks 30 may be secured to or formed into or on the housings 40 of the flow generator 12 and/or the module 14. Typically, the interlocks 30 are integral with or secured to the housings 40 of the associated flow generator 12 or module 14 as is generally illustrated throughout the Figures. The interlocks 30 may be mechanical, electromechanical, magnetic or otherwise configured to permit adjacent modules 14 or a module 14 and the flow generator 12 to be releasably secured to one another. In one aspect, the interlocks 30 may include a male interlock 32 configured to be paired with and slidably engage a female interconnect 34 on an adjacent module 14 or flow generator 12. The male interlock 32 and the female interlock 34 being configured to mechanically releasably secure the adjacent modules 14 or the flow generator 12 and an adjacent module 14.

The flow generator 12 is generally configured to generate a flow of pressurized air that may be communicated to the airways of the user. The flow generator 12 typically includes a blower 62, a blower motor 72, and a microcontroller 106. Various sensors may also be provided to monitor mechanical and/or electrical conditions. The microcontroller 106 may regulate the operation of the flow generator 12 as well as the various modules 14 secured to the flow generator 12. In addition, the microcontroller 106 may receive, process, and/or store data from the various sensors. The flow generator 12 will also typically have a series of controls 42 on the secured to the housing 40 to receive input from the user, physician, or programmer. A display 44 may also be provided to display the current status of operation and other pertinent information to the user or physician. The display 44 may be secured to the flow generator 12 or otherwise secured to the respiratory therapy apparatus to be visible to the user. In some aspects, the display 44 displays an indicator 45 indicative of the at least one module 14 secured to the flow generator 12 in a particular configuration of the respiratory therapy apparatus 10. The display 44 may at least temporarily indicate the connection or proper connection of various modules during a start up procedures or otherwise during operation of the device. The display 44 may also communicate information on the status or modes of various modules 14. This information may be provided temporarily by the display during a start up procedure or otherwise during operation, on demand by a user, such as, for example when a status button is depressed, or continuously during operation of the respiratory therapy apparatus 10. The information displayed on the display 44 may vary with various modules 14. For example, a humidifier module 16 may provide information regarding the mode of operation such as low, medium or high humidification and/or it may indicate the condition of the status of the module such as low reservoir volume to be displayed on display 44. The battery module 20 may provide information on the mode of operation such as back-up or primary power source and/or may indicate the level of charge in the associated battery or batteries to be displayed on display 44. The data transfer module 18 may provide information on the mode of operation such as receiving or sending data or may provide information on the status such as memory remaining, connectivity information, to data card, internet, computer or otherwise. Various other indications representative of the status, modes, or otherwise of the associated modules may be displayed on display 44 as will be recognized by those skilled in the art upon review of the present disclosure.

Additionally, the display 44 and user interface controls 42 may be used to setup any of the modules 14 for particular operation such as setting the humidifier module 16 to operate at any of the selectable humidification levels such as low, medium, or high humidity levels. Similarly, the display 44 and interface controls 42 may be used to command a transfer of data from the flow generator 12 to the data transfer module 18 or to allow the data transfer module 18 to be erased or overwritten with new data. The battery module 20 may be setup to use one of several possible power management schemes such as disabling the humidifier module 16 once battery power reached a certain threshold of remaining power. Numerous other setup scenarios would be recognized by those skilled in the art upon review of this disclosure.

When a microcontroller 106 is utilized by the flow generator 12, the microcontroller 106 may include a clock 124, a CPU 122, and memory 116 and may be otherwise configured as would be recognized by those skilled in the art upon review of this disclosure. The memory 116 may be used, inter alia, to store events such as usage of the flow generator 12 including ramp-up, delivery of therapeutic flow, and pauses during flow generator use. The events stored in the memory 116 may also include details of the delivery of the pressurized therapy such as pressure and flow rate, as well as various physiological parameters such as breathing rate, heart rate, and episodes of sleep disorders. The memory 116 may store the corresponding times and dates of the events to create a record of events. The record of events stored in memory 116 may extend over a time period of weeks or months and the record of events may be configured such that new events overwrite the oldest events in the record of events. The microcontroller 106 may communicate information including the record of events to the data transfer module 18 and may receive information from the data transfer module 188 via the communication interface 130. In various aspects, the microcontroller 106 may detect the modules 14 included in a particular configuration of the respiratory therapy apparatus 10 and may cause the display 44 to indicate the modules 14 included in the particular configuration.

One or more modules 14 may be configured to at least communicate data to the flow generator 12 and may be configured to receive data from the flow generator 12. In one aspect, communication may be accomplished using a wireless system. In another aspect, the adjacent modules 14 or the flow generator 12 and an adjacent module 14 may be connected by a mechanical interface 132 to permit communication between one or more modules 14 and the flow generator 12 and/or between the adjacent modules 14.

The flow generator 12 and each module 14 may include a communication interface 130 that allows the flow generator 12 to communicate to the module 14 and may allow the module 14 to communicate to the other modules 14 that may be included in a particular configuration of the respiratory therapy apparatus 10. Communication may include the ability to transfer power between modules 14, the ability to pass digital data between modules 14, the ability to transfer analog data between modules 14, and the ability of one module 14 to control another module 14.

The communication interface 130 may be, at least in part, a wireless system, which may include a radiofrequency interface 170, and optical interface 150, or combinations thereof. The radiofrequency interface 170 may include a wide range of wireless hardware and protocols to generate and receive communications by radiofrequency signal. This radiofrequency interface 170 will typically include a radiofrequency transmitter to generate the radiofrequency signal 172 and receiver to receive the radiofrequency signal 172. The radiofrequency interface 170 may employ one or more of Wireless Fidelity (WiFi), Worldwide Interoperability for Microwave Access (WiMax), Bluetooth®, and cellular protocols such as Code Division Multiple Access (CDMA) among other protocols and methodologies as will be recognized by those skilled in the art upon review of the present disclosure. The radiofrequency interface 170 may conform to IEEE 802.11 standards.

The optical interface 150 may generate and/or receive optical signals 168. The optical interface 150 may communicate using infrared wireless communication such as that defined by the specifications provided by the Infrared Data Association (IrDA) or may utilize other light-based techniques and employing such communication protocols as will be recognized by those skilled in the art upon review of this disclosure.

The communication interface 130 may include, at least in part, a mechanical interface 132 to communicate data from module 14 to module 14 via one or more channels 134. In the mechanical interface 132, one or more electrical connectors 50 including wires, pins, spring connectors, or similar or combinations thereof may be provided on flow generator 12 and modules 14. A channel 134, meaning an electrical pathway between the modules 14, may be made by bringing various contacts 50 of adjacent electrical connectors 50 into contact with one another. The electrical connector 50 may be integrated into the housing 40 or may be secured to the housing 40. Typically, the electrical connector 50 is positioned on the surface of the module 14 or flow generator 12 which abuts or is adjacent to the surface of the module 14 on which the electrical connection is to be made. In one aspect, the one or more electrical connectors 50 may be integral with an interlock 30. In other aspects, one or more electrical connectors 50 may be independent of interlock 30.

The channel 134 may be static in that the channel 134 always conveys the same type of communication. For example, a static channel 134 may always convey power. Alternatively, a channel 134 may be dynamic in that the channel 134 can convey at least a first type of communication and a second type of communication. For example, in some configurations, the channel 134 may convey power, and, in other configurations, the channel 134 may convey digital data. Communications could be overlaid on the channel 134. For example, the channel could convey power and could simultaneously convey digital information.

In various aspects, the respiratory therapy apparatus 10 may include a humidifier module 16, a data transfer module 18, and a battery module 20. The humidifier module 16 may add moisture and/or therapeutic agents to the pressurized air delivered to the user. The humidifier module 16 generally includes a reservoir 36 and a heater 112. The humidifier module 16 may be a unitary or a multi-component module. As generally illustrated throughout the Figures for exemplary purposes, the humidifier module 16 includes a multi-piece housing permitting the removal of the reservoir 36 for refilling without removal of the portion of the housing secured to the flow generator 12 by the interlock 30. The reservoir 36 contains water and/or therapeutic agents to be introduced into the user's therapy. The heater 112 heats the water and/or therapeutic agents in the reservoir 36. The heating may enhance the vaporization of the water and/or therapeutic agents. The humidifier module 16 includes at least one interlock 30 and may include an electrical connector 50. The interlock 30 and the electrical connector 50 may be integral or secured to the housing of the humidifier module 16.

The data transfer module 18 may be configured to obtain the record of events from the memory 116 over a communication interface 130. The data transfer module 18 may then transfer the record of events to some portable media 126 such as a data card or a flash memory. The portable media 126 containing the record of events may be physically sent to a health care provider who could, for example, examine the record of events and make adjustments to the therapy delivered to the user by the flow generator 12 based on the record of events. Alternatively, the data transfer module 18 may transmit the record of events over a network such as the Internet or cellular telephone network to the health care provider. The data transfer module 18 includes at least one interlock 30 and may include an electrical connector 50. The interlock 30 and the electrical connector 50 may be integral to or secured to the housing 40 of the data transfer module 18.

The battery module 20 may provide electrical power to the flow generator 12 and other modules 14 included in the respiratory therapy apparatus 10. The battery module 20 may be directly connected to an external power source. The external power source may maintain the battery within the battery module 20 in a fully charged condition. If the external power source is not provided or fails, the battery module 20 may power the flow generator 12 and other modules 14 until power from the external power source is regained or the battery of the battery module 20 has discharged. The battery module 20 includes at least one interlock 30 and may include an electrical connector 50. The interlock 30 and the electrical connector 50 may be integral or secured to the housing 40 of the battery module 20.

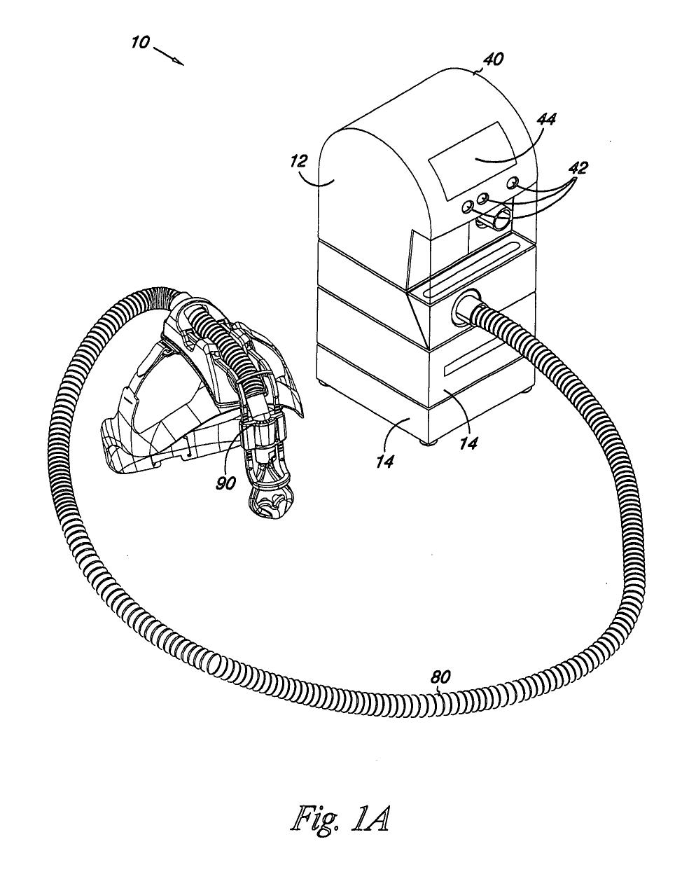

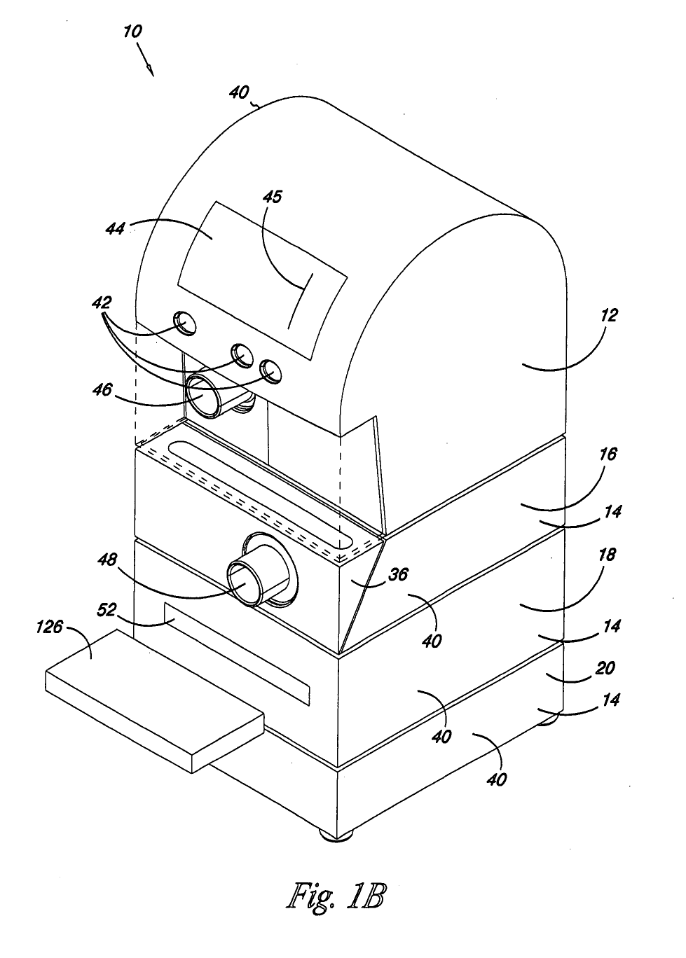

An embodiment of the respirator therapy apparatus 10 is illustrated in FIG. 1A. As illustrated, the respiratory therapy apparatus 10 includes the flow generator 12 and modules 14 in a stacked vertical configuration. The respiratory therapy apparatus, in this embodiment, includes the delivery tube 80 and user interface 90 that cooperate with the flow generator 12 to communicate pressurized air generated by the flow generator 12 to the user for inhalation.

As particularly illustrated in the embodiment of FIG. 1B, the respiratory therapy apparatus 10 may include the flow generator 12, the humidifier module 16, the data transfer module 18, and the battery module 20 stacked in a vertical configuration. The flow generator 12, in this embodiment, includes the display 44, which displays an indicator 45 indicative of the modules 14 secured to the flow generator 12 in this configuration of the respiratory therapy apparatus 10. The flow generator 12 includes an outlet 46 in fluid communication with a reservoir 36 of the humidifier module 16. For exemplary purposes, a portion of the humidifier module 16 is illustrated in phantom to show the relationship of the outlet 46 of the flow generator 12 to the humidifier module 16. As illustrated, pressurized air from the flow generator 12 is directed by the humidifier module 16 from the outlet 46 into the reservoir 36. After humidification and/or addition of therapeutic agent, the pressurized air may be directed from the humidifier module 16 through a humidifier outlet 48 to the user. A microcontroller 106 within the flow generator 12 may monitor and control the humidification of pressurized air passing through the humidifier module 16.

The data transfer module 18 may be secured inferior to the humidifier module 16. The illustrated data transfer module 18 includes a slot 52 to receive the portable media 126 to which the data transfer module 18 will transfer data indicative of the therapy delivered to the user. Typically, the data will be transferred from the memory 116 of the microcontroller 106. The illustrated battery 20 module is secured inferior to the data transfer module 18 as one embodiment. The battery module 20 provides power to at least the blower motor to provide therapy to a user when battery power is needed. The battery module 20 may be in communication with at least the microcontroller 106 of the flow generator to confer data indicative of the condition of the available power to the microcontroller 106.

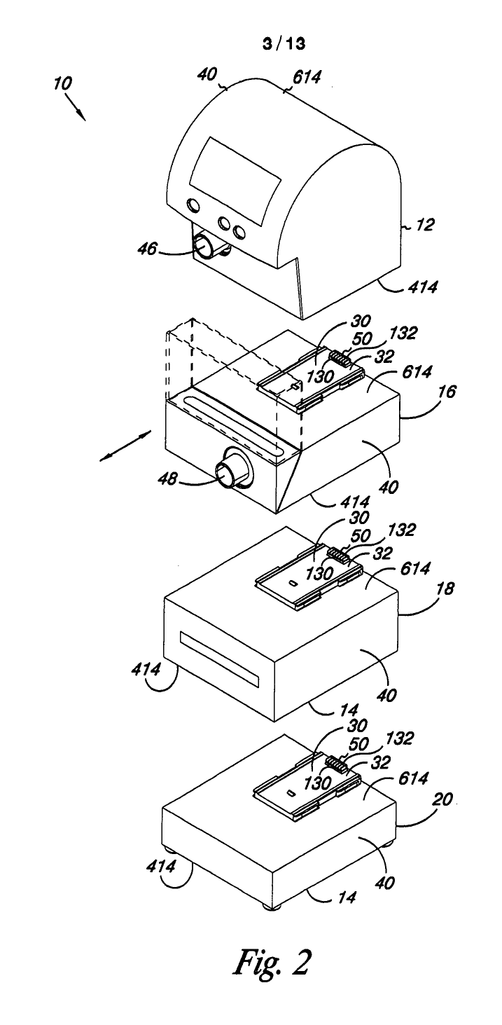

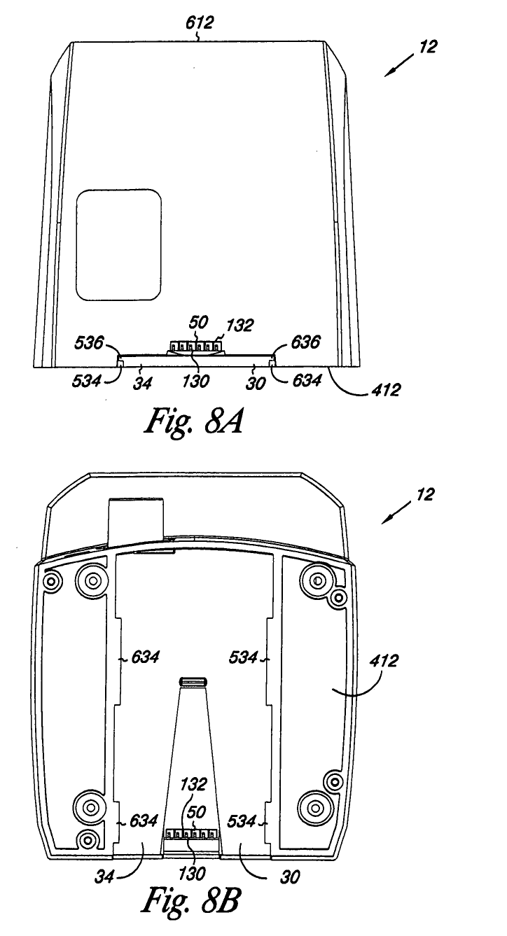

FIG. 2 particularly illustrates the embodiment of FIG. 1 in an exploded configuration. In the exploded configuration, interlocks 30 are shown as molded into an upper surface 614 of the modules 14. The interlocks 30 on the upper surface 614 are configured to releasably engage interlocks 30 (as, for example, shown in FIGS. 8A and 8B) on the lower surface 414 of the module 14 in an immediately superior position to releasably secure adjacent modules 14 or releasably secure the flow generator 12 to an adjacent module 14. The most superior module 614 is secured to an interlock 30 on the lower surface 412 of the flow generator 12. As illustrated in one embodiment, interlocks 30 on the upper surfaces 614 of the modules 14 are configured as male interlocks 32. Accordingly, the interlocks 30 (as, for example, shown in FIGS. 8A and 8B) on the lower surface 412 of the flow generator 12 and the lower surface 414 of the modules are configured as female interlocks 34 or otherwise configured to releasably engage male interlocks 32. As illustrated for exemplary purposes, the female interlock 34 of a superiorly positioned module 14 or flow generator 12 is configured to receive the male interlock 32 of an inferiorly positioned module 14 and to releasably engage one another by sliding the superior module 14 or flow generator 12 rearward over the inferior module 14. Each of the illustrated male interlocks 32 includes an electrical connector 50. In one embodiment, the electrical connectors 50 are secured to the male interlocks 32. The electrical connectors 50 are illustrated as six pin connectors for exemplary purposes. The electrical connectors 50 of the male interlocks 32 are configured to be placed in electrical contact with the contacts of a corresponding electrical connector 50 positioned within the female interlock 34.

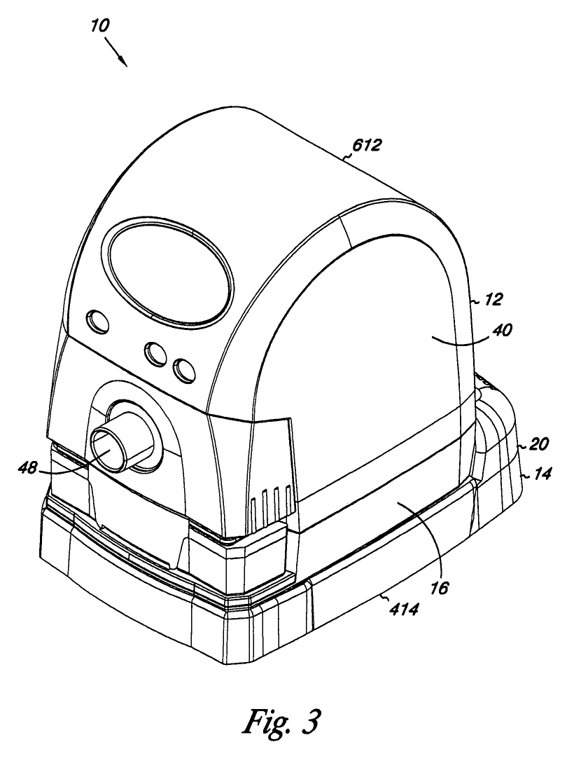

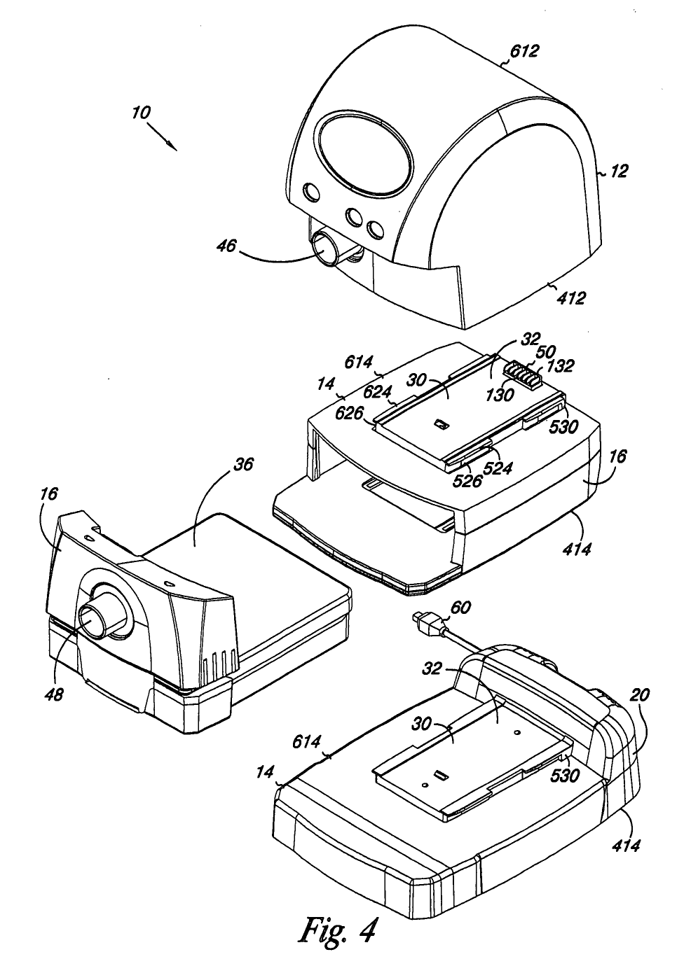

FIGS. 3 and 4 particularly illustrate another embodiment of the respiratory therapy apparatus 10 in accordance with one or more of the present inventions. As illustrated in FIG. 3, the respiratory therapy apparatus 10 includes the flow generator 12 and two modules 14 stacked in a vertical configuration. The two modules 14 include the humidifier module 16 and the battery module 20. The humidifier module 16 is releasably secured in an inferior position to the flow generator 12 for exemplary purposes. The battery module 20 is releasably secured in an inferior position to the humidifier module 16, again, for exemplary purposes. FIG. 4 illustrates an exploded view of the apparatus of FIG. 3. Again, interlocks 30 on the upper surface 614 are configured to releasably engage interlocks 30 (as, for example, shown in FIGS. 8A and 8B) on the lower surface 414 of the module 14 in an immediately superior position. The most superior upper surface 614 is secured to the interlock 30 on the lower surface 412 of the flow generator 12. As illustrated, interlocks 30 on the upper surfaces 614 of the modules 14 are configured as male interlocks 32. Accordingly, the interlocks 30 (as, for example, shown in FIGS. 8A and 8B) on the lower surface 412 of the flow generator 12 and the lower surface 414 of the module 16 may be configured as female interlocks 34 or otherwise configured to be releasably engage to male interlocks 32. As illustrated, the male interlock 32 associated with the humidifier module 16 includes an electrical connector 50. However, the male interlock 32 associated with the battery module 20 does not, as particularly illustrated, include an electrical connector 50. Alternatively, a cable 60 has been provided in the illustrated embodiment to communicate electrical current to the flow generator 12.

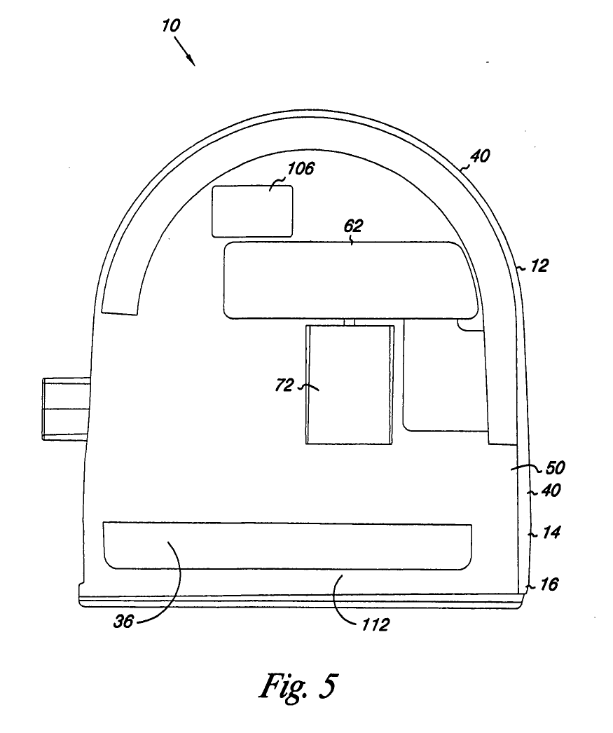

FIG. 5 illustrates a cross-sectional side view of an embodiment of the respiratory therapy apparatus 10 including a flow generator 12 and a humidifier module 16 stacked in a vertical configuration. The cross-section shows the blower 62 and blower motor 72 as well as a microcontroller 106 operably secured within housing 40 of the flow generator 12. The microcontroller 106 may communicate with the blower motor 72 and various sensors within the flow generator 12 to control the operation of the blower motor 72 and to receive data from the blower motor 72 and the sensors. The microcontroller 106 is further in electrical communication with the electrical connector 50 of the flow generator 12. With the housing 40 of the humidifier module 16 secured to the flow generator 12 by male interlock 32 and female interlock 34, the electrical connector 50 of the flow generator 12 is placed in electrical contact with the electrical connector 50 of the humidifier module 16. The microcontroller 106 may receive data indicative of temperature of fluid in the reservoir 36 and regulate the heater 112 to maintain the fluid at the proper temperature.

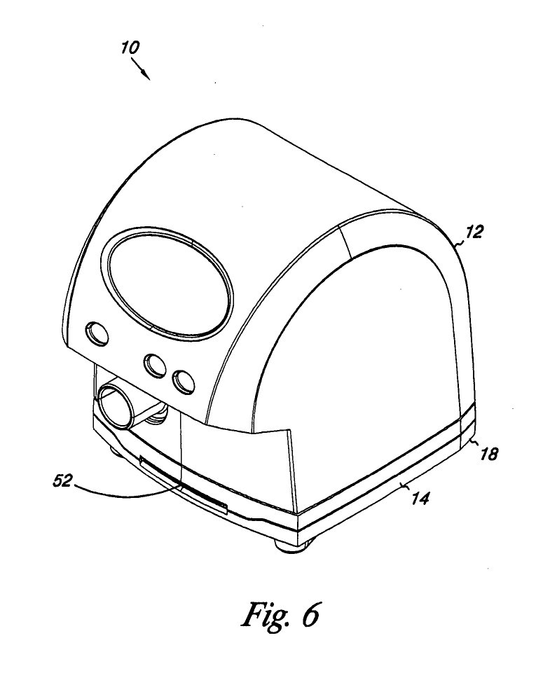

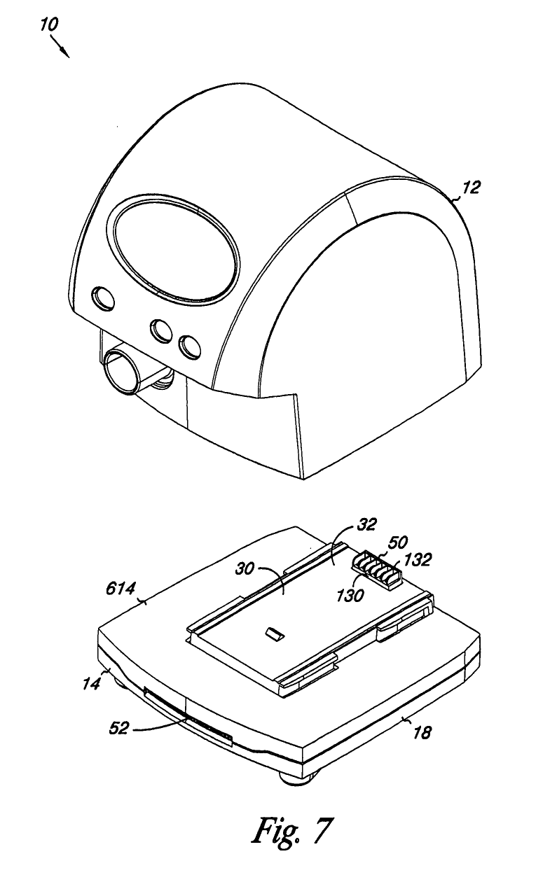

FIGS. 6 and 7 particularly illustrate another embodiment of the respiratory therapy apparatus 10 in accordance with one or more of the present inventions. As illustrated in FIG. 6, the respiratory therapy apparatus 10 includes a flow generator 12 and a data transfer module stacked in a vertical configuration. The data transfer module 18 is releasably secured inferior to the flow generator 12. The illustrated data transfer module 18 includes a slot 52 to receive a portable media 126 to which the data transfer module 18 will transfer data indicative of the therapy delivered to the user. Typically, the data will be transferred from the memory 116 of the microcontroller 106 by the data transfer module 18. In the exploded configuration of FIG. 7, an interlock 30 is shown as molded into an upper surface 614 of the data transfer module 18. The interlock 30 on the upper surface 614 of the data transfer module 18 is configured to releasably engage interlock 30 (as, for example, shown in FIGS. 8A and 8B) on the lower surface 412 of the flow generator 12. As illustrated, interlock 30 on the upper surfaces 614 of data transfer module 18 is configured as male interlock 32. Accordingly, the interlock 30 (as, for example, shown in FIGS. 8A and 8B) on the lower surface 412 of the flow generator 12 configured as female interlock 34 or is otherwise configured to releasably engage a male interlock 32. The illustrated male interlock 32 includes an electrical connector 50. The electrical connector 50 is positioned on a surface of the male interlock 32. The electrical connector 50 is illustrated as a six pin connector for exemplary purposes. The illustrated electrical connector 50 of the male interlock 32 is configured to be place in electrical contact with the corresponding contacts on an electrical connector 50 positioned within or about the female interlock 34.

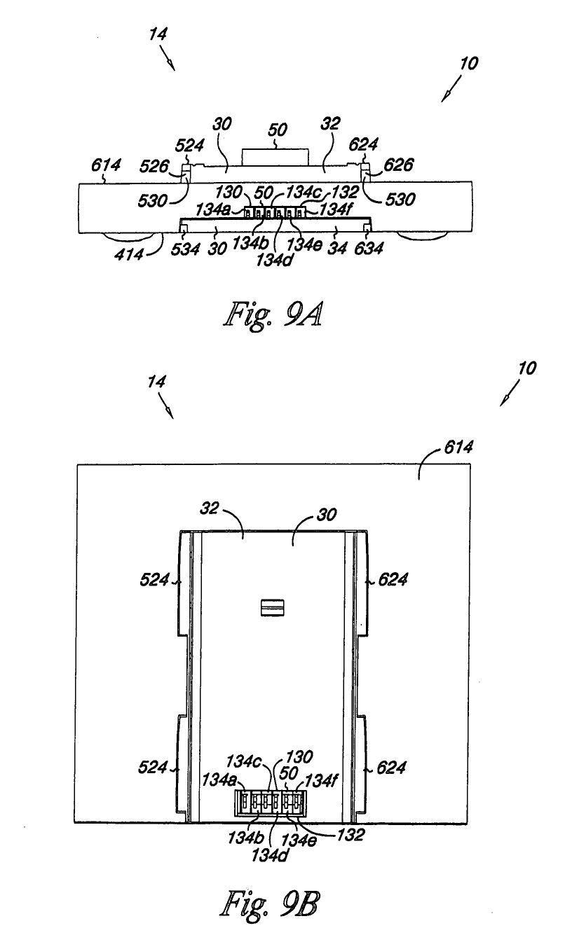

FIGS. 8A, 8B, 9A and 9B illustrate some exemplary details of embodiments for interlocks 30 for removably securing a flow generator 12 to one or more modules 14 in accordance with one or more of the present inventions. As illustrated in FIGS. 9A and 9B, the module 14 includes a male interlock 32 on an upper surface 614. As illustrated in FIGS. 8A, 8B, and 9A, the flow generator 12 and module 14 include a female interlock 34 on a lower surface 412 and 414, respectively. The female interlock 34 includes a cavity with a first inward facing flange 534 and a second inward facing flange 634. The first inward facing flange 534 and the second inward facing flange 634 and the adjacent housing 40 of the flow generator 12 and the module 14 define a first channel 536 and a second channel 636 extending from the back side of the flow generator 12 and the module 14 toward the front side. The male interlock 32 is a protuberance extending upward from the body 40 of the module 14. The protuberance includes a first laterally extending flange 524 and a second laterally extending flange 624. The first laterally extending flange 524 and the second laterally extending flange 624 and the adjacent housing 40 of the module 14 define a first lateral channel 526 and a second lateral channel 626 (both shown in phantom) extending from the front side of the module 14 toward the back side of the module 14. As illustrated, two first laterally extending flanges 524 and two second laterally extending flanges 624 are provided on the upwardly extending protuberance of module 14. Each of the illustrated laterally extending flanges 524, 624 includes a posterior stop 530 to limit the forward movement of the male interlock 32 into the female interlock 34. As illustrated, the male interlock 32 of the module 14 is configured to upwardly engage the female interlock 34 on the lower surface 412 of the flow generator 12 and then to slide forward until the electrical contacts 50 of the flow generator 12 and the module 14 engage one another in electrical contact. In another aspect, the male interlock 32 having a single first laterally extending flange 524 and a single second laterally extending flange 624 could be configured to engage the female interlock 34 from a rearward position relative to the flow generator 12 and then to slide forward until the electrical contacts 50 of the flow generator 12 and the module 14 engage one another in electrical contact. The illustrated communication interface 130 is configured as a mechanical interface 132 having six channels 134a, 134b, 134c, 134d, 134e, 134f in the particular embodiment illustrated in FIGS. 9A and 9B. Those skilled in the art will recognize upon review of this disclosure that the mechanical interface 132 may be configured in a variety of ways and may be configured to include various numbers of channels 134.

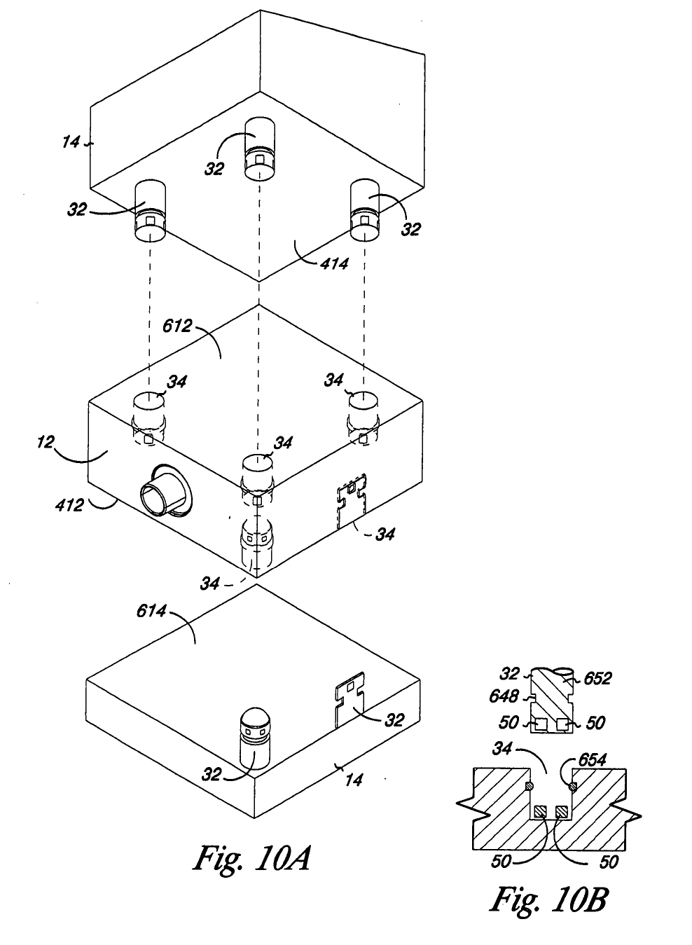

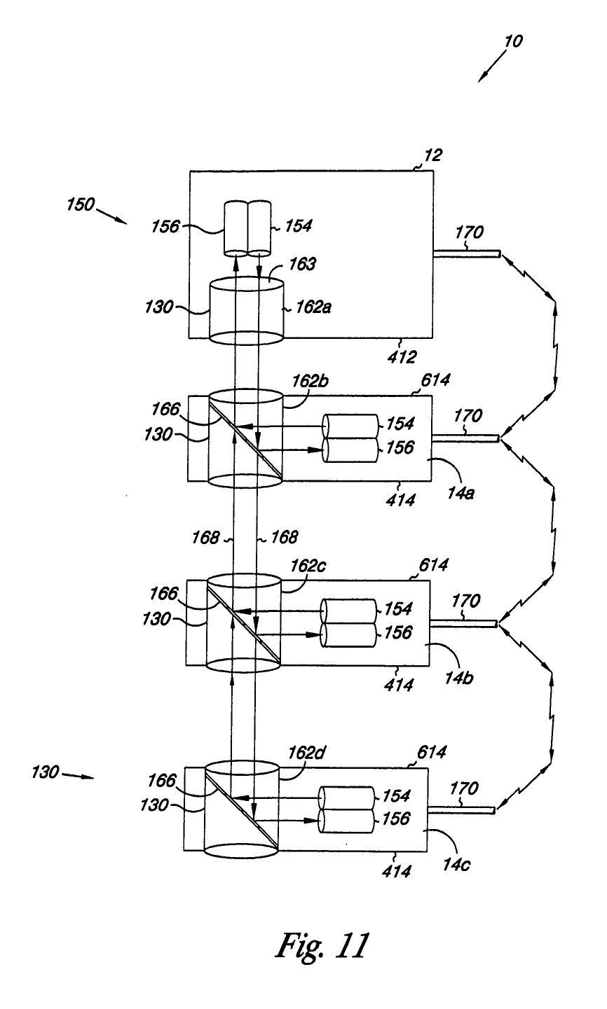

FIGS. 10A and 10B illustrate some exemplary details of an embodiment for male interlocks 32 and female interlocks 34 for releasably securing one or more modules 14 to a flow generator 12 in accordance with one or more of the present inventions. In this exemplary embodiment, a module 14 is illustrated as positioned superior to the flow generator 12 and with lower surface 414 oriented such that male interlocks 32 extending from the lower surface 414 mate with corresponding female interlocks 34 on the upper surface 612 of the flow generator 12.

A second module 14 positioned inferior to the flow generator is also included in the embodiment illustrated in FIG. 10A. Male interlocks 32 extend from the upper surface 614 of module 14 and the inferiorly positioned module is oriented so that the male interlocks 32 releasably engage with female interlocks 34 on the lower surface 412 of the flow generator. A male interlock 32 of the lowermost module 14 is shown peripherally positioned on the lowermost module 14 to engage a female interlock 34 formed on the side of the superiorly positioned flow generator 12 for exemplary purposes. Thus, in this embodiment, the flow generator may be stacked between two modules 14, with one module 14 placed superior to the flow generator 12 and one module 14 placed inferior to flow generator 12. Those skilled in the art upon review of this disclosure will recognize that the respiratory therapy apparatus 10 in various embodiments may incorporate a variety of modules 14 and that the modules 14 may be stacked in a variety of combinations with respect to one another and with respect to the flow generator 12.

The male interlocks 32 in the embodiment illustrated in FIGS. 10A and 10B is generally configured as circumferentially slotted 648 cylindrical members 652. One or more electrical connectors 50 may be located in the ends of cylindrical members 652 as shown, interposed about the circumference of the cylindrical members 652 (not shown), or otherwise disposed about the cylindrical members 652. The male interlocks 32 are configured to be received within corresponding female interlocks 34, and the female interlocks 34 are configured to so receive the male interlocks 32. The female interlock 34, in this embodiment, includes a rubber gasket 654. When the male interlock 32 is received within the female interlock 34, the rubber gasket 654 is configured to be received within the circumferential slot 648 to releasably engage the male interlock 32 within the female interlock 34 by compressionally engaging the inner surface of female interlock 34. For exemplary purposes, FIG. 10B illustrates electrical connectors 50 interposed circumferentially around the female interlock 34 to contact the electrical connectors 50 interposed about the circumference of the cylindrical member 652 of the male interlock 34 to form an electrical connection between the module 14 and the flow generator 12.

FIG. 11 illustrates another embodiment of the flow generator 12 and modules 14a, 14b, 14c in a stacked vertical configuration. As illustrated, the lower surface 412 of the flow generator 12 may be generally biased against the upper surface 614 of module 14a. The lower surface 414 of module 14a may be biased against the upper surface 614 of module 14b, and the lower surface 414 of module 14b may, in turn, rest upon the upper surface 614 of module 14c. The lower surface 414 of module 14c, may rest upon a table, nightstand, or other surface.

As illustrated in FIG. 11, the flow generator 12 and modules 14a, 14b, 14c include a communication interface 130 configured for optical communication by optical signal 168. The flow generator 12 and modules 14a, 14b, 14c include light tube elements 162a, 162b, 162c, 162d. The light tube elements 162a, 162b, 162c, 162d may be aligned to form a light tube 160 when the flow generator 12 and modules 14a, 14b, 14c are in a stacked vertical configuration. The light tube elements 162a, 162b, 162c, 162d may be variously configured to align such as, for example, by including corresponding male/female ends. The light tube 160 defines a light tube passage 163 configured to allow the passage of the optical signal 168.

The flow generator 12 and the modules 14a, 14b, 14c each include an emitter 154 to transmit communications by optical signal 168 and a detector 156 to receive communications by optical signal 168. As illustrated, the light tube elements 162b, 162c, 162d on modules 14a, 14b, 14c may include beam splitters 166 to deflect a portion of the optical signal 168 to the detectors 156 while allowing a portion of the optical signal 168 to pass through the light tube passage 163. The beam splitters 166 may also deflect the optical signal 168 from the emitter 154 into the light tube passage 163. The beam splitters 166 may be configured, for example, as a ½ silvered mirrors.

The flow generator 12 and module 14a, as illustrate in FIG. 11, may also include the communication interface 130 configured to include the radiofrequency interface 170 to allow communication between flow generator 12 and module 14a by radiofrequency signal 172.

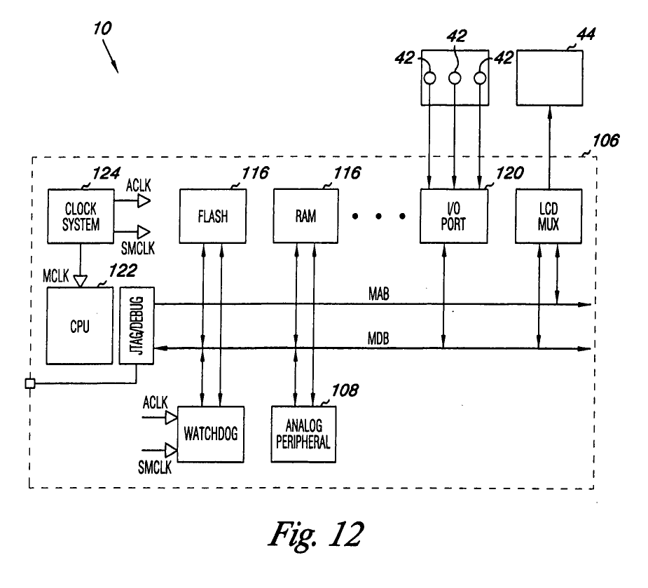

FIG. 12 illustrates an embodiment of the microcontroller 106. In this embodiment, the microcontroller 106 includes CPU 122, clock 124, and memory 116. The memory 116, in this embodiment, includes both flash memory and RAM. The microcontroller is configured to communicate with analog peripheral 108 and may communicate with I/O port 120. The I/O port 120 may be included in the data transfer module 18. The I/O port 120 may be configured to transfer digital data to and/or from the portable media 126. The microcontroller 106, as illustrated, may communicate with the control 42 secured to housing 40 and may also communicate with display 44. In various embodiments, the microcontroller 106 may detect the modules 14 included in the particular configuration of the respiratory therapy apparatus 10 and may indicate the modules 14 on the display 44.

The present inventions also provide methods for modularly configuring the respiratory therapy apparatus 10. The methods may include providing a flow generator 12 and a module 14, and securing the module 14 to the flow generator 12 in a vertical stacked orientation. The methods may further include providing a plurality of modules 14 and securing the modules 14 to one another and to the flow generator 12 in a vertical stacked orientation. The methods may include providing an interlock 30 on the lower surface 412 of the flow generator 12 and providing an interlock 30 on the upper surface 614 of the module 14, and using the interlocks 30 to removably secure the upper surface 614 of the module 14 to the lower surface 412 of the flow generator 12. The methods may further include providing interlocks 30 on lower surfaces 414 of one or more modules 14 and securing the modules 14 by the interlocks 30 in a stacked vertical configuration. The methods may include configuring the interlocks 30 as male interlocks 32 and female interlocks 34.

The methods may further include one or more of the following steps in various orders as will be recognized by those skilled in the art upon review of the present disclosure. A step of providing a module 14 having a first interlock 30 and a flow generator 12 having a second interlock 30. A step of releasably engaging the first interlock 30 to the second interlock 30 to releasably secure the module 14 to the flow generator 12 to form a respiratory therapy apparatus 10. A step of releasably coupling a first communication interface 130 of a first module 14 or a flow generator 12 to the second communication interface 130 of a second module 14 to communicate at least one of data or power between the first communication interface and the second communication interface. A step of having the user connect at least the flow generator 12 to a power supply such as a wall outlet. A step of securing a delivery tube 80 with the flow generator 12. A step of selecting the parameters for positive airway pressure therapy. A step of positioning a user interface 90 such that the user interface is in communication with the airways of the user. A step of initiating a positive pressure airway therapy. A step of administering a positive airway pressure therapy to the user with the respiratory therapy apparatus 10. One or more of the preceding steps may be taken by a user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A respiratory therapy apparatus, comprising:
a flow generator configured to provide a positive airway pressure therapy to a user, the flow generator including a microcontroller, a blower, a blower motor, and a first interlock, wherein the microcontroller regulates the operation of at least the blower motor; and,
at least one module including a second interlock, the second interlock releasably engaged with the first interlock to releasably secure the flow generator to the at least one module in a stacked vertical configuration.

2. The respiratory therapy apparatus, as in claim 1, further comprising the first interlock comprising a female interlock and the second interlock comprising a male interlock.

3. The respiratory therapy apparatus, as in claim 2, further comprising the female interlock including a first electrical connector and the male interlock including a second electrical connector, the first electrical connector and the second electrical connector configured to electrically contact one another when the first interlock is releasably engaged with the second interlock.

4. The respiratory therapy apparatus, as in claim 1, further comprising the flow generator and the at least one module stacked in a vertical configuration where a lower surface of the at least one module positioned adjacent to an upper surface of the flow generator.

5. The respiratory therapy apparatus, as in claim 1, further comprising a display, the display secured to the flow generator, the display at least temporarily displays an indicator indicative of the at least one module releasably secured to the flow generator.

6. The respiratory therapy apparatus, as in claim 1, further comprising a display, the display secured to the flow generator, the display at least temporarily displays an indicator indicative of the status of at least one module releasably secured to the flow generator.

7. The respiratory therapy apparatus, as in claim 1, further comprising a display, the display secured to the flow generator, the display at least temporarily displays an indicator indicative of the mode of at least one module releasably secured to the flow generator.

8. The respiratory therapy apparatus, as in claim 1, further comprising:
the flow generator in communication with the at least one module by a communication interface.

9. The respiratory therapy apparatus, as in claim 6, further comprising:
the communication interface including a hardware interface.

10. The respiratory therapy apparatus, as in claim 6, further comprising:
the communication interface including an optical interface.

11. The respiratory therapy apparatus, as in claim 6, further comprising:
the communication interface includes a radiofrequency interface.

12. A respiratory therapy apparatus, comprising:
a flow generator; and
a plurality of modules, the modules removably securable to one another, the modules removably securable to the flow generator in a plurality of stacked vertical configurations.

13. A method, comprising:
providing a flow generator and a module; and
and releasably securing the module to the flow generator in a vertical stacked orientation.

14. A method, as in claim 13, further comprising releasably coupling a first communication interface of the flow generator to a second communication interface of the module to communicate at least one of data or power between the first communication interface and the second communication interface.

15. A method, as in claim 13, further comprising releasably engaging a first interlock of the flow generator to a second interlock of the module to releasably secure the module to the flow generator.

16. A method, as in claim 15, further comprising releasably coupling a first communication interface of the flow generator to a second communication interface of the module to communicate at least one of data or power between the first communication interface and the second communication interface.
